(19) United States
(12) Patent Application Publication
(10) Pub. No.: US 2026/0295281 A1
(43) Pub. Date: Oct. 1, 2026

(54) FIXATION MESH FOR IMPLANTABLE MEDICAL DEVICE

(71) Applicant: Medtronic, Inc., Minneapolis, MN (US)

(72) Inventors: Thomas A. Anderson, New Hope, MN (US); Melissa G.T. Christie, Ham Lake, MN (US)

(21) Appl. No.: 19/477,970

(22) PCT Filed: Mar. 25, 2024
(86) PCT No.: PCT/IB2024/052846

§ 371 Date: Oct. 23, 2025

Related U.S. Application Data
(60) Provisional application No. 63/497,896, filed on Apr. 24, 2023.

(51) Int. Cl. A61N 1/375; A61N 1/05

(52) U.S. Cl. CPC A61N 1/37518; A61N 1/0573; A61N 1/37512

(57) ABSTRACT

A device comprising: an elongated housing extending from a proximal end to a distal end, the elongated housing being configured to be implanted wholly within a chamber of a heart; a first electrode extending distally from the distal end of the elongated housing, the first electrode comprising an elongated body defining a helix; a second electrode disposed on the distal end of the elongated housing, wherein the second electrode is configured to be placed in contact with wall tissue of the chamber without penetrating the wall tissue; and a mesh disposed over at least a portion of a distal portion of the elongated housing, wherein the distal portion of the elongated housing defines the distal end, wherein the mesh comprises a plurality of filaments, and wherein the mesh is configured to facilitate growth of the wall tissue around one or more filaments of the plurality of filaments.

This application claims the benefit of U.S. Provisional Patent Application Ser. No. 63/497,896, filed Apr. 24, 2023, the entire content of which is incorporated herein by reference.

### TECHNICAL FIELD

The disclosure relates to medical devices, and more particularly to fixation mechanisms of medical devices.

### BACKGROUND

Various types of implantable medical devices (IMDs) have been implanted for treating or monitoring one or more conditions of a patient. Such IMDs may be adapted to monitor or treat conditions or functions relating to heart, muscle, nerve, brain, stomach, endocrine organs or other organs and their related functions. Such IMDs may be associated with leads that position electrodes at a desired location or may be leadless with electrodes integrated with and/or attached to the device housing. These IMDs may have the ability to wirelessly transmit data either to another device implanted in the patient or to another instrument located externally of the patient, or both.

A cardiac pacemaker is an IMD configured to deliver cardiac pacing therapy to restore a more normal heart rhythm. Such IMDs sense the electrical activity of the heart, and deliver cardiac pacing based on the sensed electrical activity, via electrodes. Some cardiac pacemakers are implanted a distance from the heart and coupled to one or more leads that intravascularly extend into the heart to position electrodes with respect to cardiac tissue. Some cardiac pacemakers are sized to be completely implanted within one of the chambers of the heart and may include electrodes integrated with or attached to the device housing rather than leads. Some cardiac pacemakers provide dual chamber functionality, by sensing and/or stimulating the activity of both atria and ventricles, or other multi-chamber functionality. A cardiac pacemaker may provide multi-chamber functionality via leads that extend to respective heart chambers, or multiple cardiac pacemakers may provide multi-chamber functionality by being implanted in respective chambers.

### SUMMARY

In general, this disclosure is directed to implantable medical devices (IMDs) configured to sense and deliver electrical signals to tissue of a patient via a plurality of electrodes at or near a distal end of an elongated housing of the IMD. More particularly, this disclosure is directed to IMDs with a fixation mesh disposed around a distal portion of the elongated housing of the IMD. The fixation mesh may facilitate growth of tissue around filaments defining the fixation mesh to affix IMD to the tissue, e.g., to inhibit unintended rotation and/or dislodgement of the IMD from the tissue.

In some examples, a single IMD is implanted in one chamber of a heart of the patient and is able to sense in and/or deliver cardiac pacing to more than one chamber, which may avoid the need for a leaded device or multiple smaller devices to provide such functionality, which may reduce the amount of material implanted within the patient. In some examples, such an implantable medical device includes a distal electrode that is configured to penetrate through wall tissue of the heart chamber in which the device is implanted, and into wall tissue of another heart chamber. In addition to the distal electrode, the device includes a reference electrode and one or more proximal electrodes configured to contact the wall tissue of the heart chamber. The distal electrode may be a helix configured to penetrate tissue of the patient. The distal electrode may be configured to sense in and/or deliver cardiac pacing to one chamber of the heart and the one or more proximal electrodes may be configured to sense in and/or deliver cardiac pacing to another separate chamber of the heart.

The IMD may include an elongated housing extending from a proximal end to a distal end. The electrodes can be connected to a distal end of the elongated housing. As described herein, the IMD may include a fixation mesh disposed around a distal portion of and/or over the distal face of the elongated housing. The fixation mesh may be defined by a plurality of interweaving filaments. The fixation mesh may be affixed to the elongated housing of IMD. The fixation mesh may allow for growth of tissue between the fixation mesh and the elongated housing and/or around the filaments of the fixation mesh, thereby affixing the IMD to the tissue. Tissue growth around the fixation mesh may improve contact between the proximal electrode and the tissue relative to another identical IMD without the fixation mesh. The improved contact may improve performance by, e.g., reducing pacing thresholds for the proximal electrode. The tissue growth within around the fixation mesh may further improve performance of the IMD by preventing unintended rotation and/or dislodgment of the IMD from tissue of the patient, thereby improving the consistency of the sensing and/or pacing capabilities of the electrodes.

The fixation mesh may include a plurality of openings configured to receive distal electrode(s), proximal electrode(s), or other features extending from the distal end of the IMD. The openings may be formed as a part of manufacturing the fixation mesh or may be formed by removing material from a pre-formed fixation mesh to define the openings. The plurality of openings may allow the fixation mesh to be disposed across the distal face of the elongated housing and may increase the contact surface area between the tissue and the fixation mesh, thereby increasing the fixation of the IMD to the tissue.

In some examples, this disclosure is directed to a device comprising: an elongated housing extending from a proximal end to a distal end, the elongated housing being configured to be implanted wholly within a chamber of a heart; a first electrode extending distally from the distal end of the elongated housing, the first electrode comprising an elongated body defining a helix; a second electrode disposed on the distal end of the elongated housing, wherein the second electrode is configured to be placed in contact with wall tissue of the chamber without penetrating the wall tissue; and a mesh disposed over at least a portion of a distal portion of the elongated housing, wherein the distal portion of the elongated housing defines the distal end, wherein the mesh comprises a plurality of filaments, and wherein the mesh is configured to facilitate growth of the wall tissue around one or more filaments of the plurality of filaments.

In some examples, this disclosure is directed to a fixation device comprising: an elongated body extending distally from a distal end of an implantable medical device, the elongated body comprising: a proximal end located at the distal end of the implantable medical device, and a helix extending distally from the proximal end and defining one or more coils, wherein a distal end of the helix is configured to penetrate into tissue of a patient; and a mesh disposed over at least a portion of a distal portion of the implantable medical device, wherein the distal portion of the implantable medical device defines the distal end of the implantable medical device, wherein the mesh comprises a plurality of filaments, and wherein the mesh is configured to facilitate growth of the tissue around one or more filaments of the plurality of filaments.

In some examples, this disclosure is directed to a method comprising: affixing a first electrode to a distal end of an elongated housing of an implantable medical device, wherein the first electrode is configured to extend distally from the distal end of the elongated housing, wherein the first electrode comprises an elongated body defining a helix, and wherein the implantable medical device is configured to be implanted wholly within a chamber of a heart; affixing a second electrode to the distal end of the elongated housing, wherein the second electrode is configured to be placed in contact with wall tissue of the chamber without penetrating the wall tissue; and affixing a mesh over a distal portion of the elongated housing, wherein the distal portion of the elongated housing defines the distal end of the elongated housing, wherein the mesh comprises a plurality of filaments, and wherein the mesh is configured to facilitate growth of the wall tissue around one or more filaments of the plurality of filaments.

In some examples, this disclosure is directed to a device comprising: an elongated housing extending from a proximal end to a distal end, the elongated housing being configured to be implanted wholly within a chamber of a heart; a fixation helix extending distally from the distal end of the elongated housing; an electrode disposed at the distal end of the elongated housing, wherein the second electrode is configured to be placed in contact with wall tissue of the chamber without penetrating the wall tissue; and a mesh disposed over at least a portion of a distal portion of the elongated housing, wherein the distal portion of the elongated housing defines the distal end of the housing, wherein the mesh comprises a plurality of filaments, and wherein the mesh is configured to facilitate growth of the wall tissue around one or more filaments of the plurality of filaments.

This summary is intended to provide an overview of the subject matter described in this disclosure. It is not intended to provide an exclusive or exhaustive explanation of the methods and systems described in detail within the accompanying drawings and description below.

### BRIEF DESCRIPTION OF DRAWINGS

The details of one or more examples of this disclosure are set forth in the accompanying drawings and the description below. Other features, objects, and advantages of this disclosure will be apparent from the description and drawings, and from the claims.

FIG. 1 is a conceptual diagram illustrating an example device implanted in the heart of a patient, in accordance with one or more aspects of this disclosure.

FIG. 2A is a perspective diagram illustrating an example configuration of the device of FIG. 1 with a fixation mesh disposed around a distal portion of the device.

FIG. 2B is a perspective diagram illustrating another example configuration of the device of FIG. 1 with a fixation mesh disposed over a distal end of the device.

FIG. 3A is a perspective diagram illustrating a top-down view of an example configuration of the device of FIG. 2B.

FIG. 3B is a perspective diagram illustrating a top-down view of another example configuration of the device of FIG. 2B.

FIG. 4A is a perspective diagram illustrating a side view of a fixation mesh disposed around a removable distal portion of the device of FIG. 2A.

FIG. 4B is a perspective diagram illustrating another example side view of a fixation mesh disposed around a removable distal portion of the device of FIG. 2A.

FIG. 4C is a perspective diagram illustrating another example side view of a fixation mesh disposed around a distal portion of the device of FIG. 2A.

FIG. 4D is a perspective diagram illustrating another example side view of a fixation mesh disposed around a distal portion of the device of FIG. 2A.

FIG. 5A is a perspective diagram illustrating an example configuration of a fixation mesh.

FIG. 5B is a cross-section diagram illustrating a cross-section view of the example configuration of the fixation mesh of FIG. 5A, the cross-section being taken along line A-A of FIG. 5A and along the length of a filament of the fixation mesh.

FIG. 6 is a conceptual diagram of the device of FIGS. 1-5B implanted at a target implant site.

FIG. 7 is a block diagram illustrating an example configuration of the device of FIG. 1.

FIG. 8 is a flowchart illustrating an example process for sensing a cardiac electrical signal and delivering cardiac pacing therapy to a heart of a patient via an example device of any of FIGS. 1-7.

### DETAILED DESCRIPTION

In general, this disclosure is directed to distal end configurations for implantable medical devices (IMDs). More particularly, this disclosure is directed to IMDs having a distal end at least partially enveloped by a fixation mesh for fixation of the IMD with respect to patient tissue, and/or a plurality of electrodes configured to sense electrical signals from and to deliver electrical stimulation (e.g., cardiac pacing) to tissue of a patient.

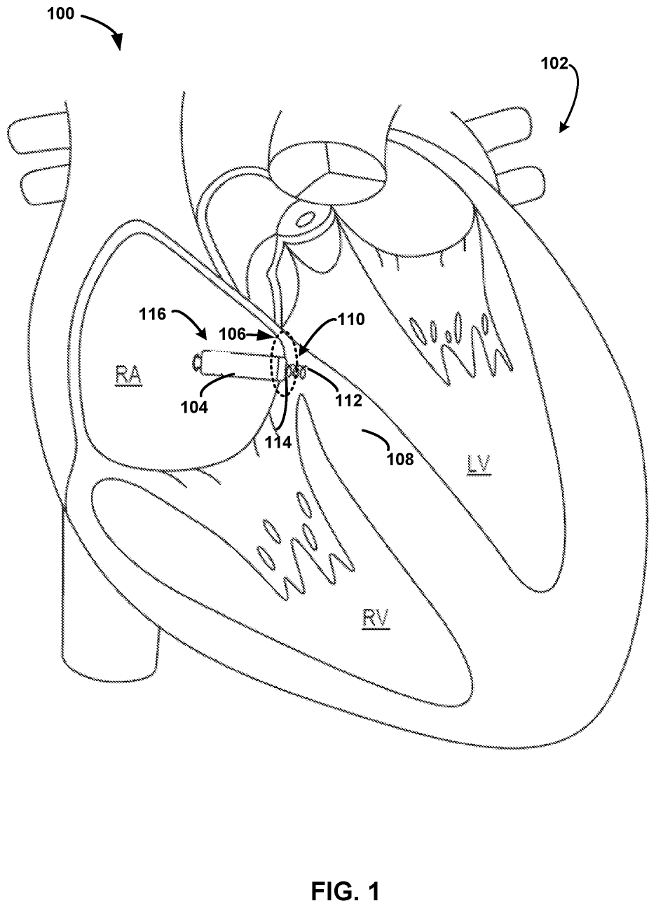

FIG. 1 is a conceptual diagram illustrating an example device 104 implanted in the heart 102 of a patient, in accordance with one or more aspects of this disclosure. Device 104 is shown implanted in the right atrium (RA) of the patient's heart 102 in a target implant region 106, such as the triangle of Koch, in heart 102 of the patient with a distal end of device 104 directed toward the left ventricle (LV) of the patient's heart 102. Although in the example of FIG. 1 the distal end of device 104 is directed toward the LV, the distal end may be directed to other targets, such as interventricular septum of heart 102. Target implant region 106 may lie between the bundle of His and the coronary sinus and may be adjacent the tricuspid valve.

Device 104 includes a distal end 110 and a proximal end 116. Distal end 110 includes a first electrode 112, and a second electrode 114. First electrode 112 may define a helical shape, e.g., as illustrated in FIG. 1. First electrode 112 extends from distal end 110 and may penetrate through the wall tissue of a first chamber (e.g., the RA in the illustrated example) into wall tissue of a second chamber (e.g., ventricular myocardium 108 of the LV in the illustrated example). Second electrode 114 may be disposed on a ramp extending distally from distal end 110 and is configured to be placed in contact with the wall tissue of the first chamber without penetration of the wall tissue of the first chamber by second electrode 114. Second electrode 114 may contact the wall tissue of the first chamber as first electrode 112 penetrates the wall tissue of the first chamber.

The configuration of electrodes 112 and 114 illustrated in FIG. 1 allows device 104 to sense cardiac signals and/or deliver cardiac pacing to multiple chambers of heart 102, e.g., the RA and ventricle(s) in the illustrated example. In this manner, the configuration of electrodes 112 and 114 may facilitate the delivery of A-V synchronous pacing by single device 104 implanted within the single chamber, e.g., the RA. While device 104 is implanted at target implant region 106 to sense in and/or pace the RA and ventricle(s) in the example shown in FIG. 1, a device having an electrode configuration in accordance with the examples of this disclosure may be implanted at any of a variety of locations to sense in and/or pace any one, two or more chambers of heart 102. For example, device 104 may be implanted at region 106 or another region, and first electrode 112 may extend into tissue, e.g., myocardial tissue, of the LV or interventricular septum to, for example, facilitate the delivery of A-V synchronous pacing. Furthermore, a device having an electrode configuration in accordance with the examples of this disclosure may be implanted at any of a variety of locations within a patient for sensing and/or delivery of therapy to other patient tissue. In some examples, first electrode 112 extends into the tissue of heart 102 at region 106 and affix device 104 to the tissue of heart 102.

Device 104 may include a fixation mesh disposed around at least a portion of distal end 110. The fixation mesh may be disposed around an outer perimeter of a distal portion of the housing of device 104 defining distal end 110 and/or may be disposed over a distal face of device 104. The fixation mesh may facilitate growth of the tissue of heart 102 around and within the fixation mesh to affix device 104 to the tissue of heart 102. The fixation mesh and the ingrown tissue may inhibit unintended rotation or dislodgment of device 104 from the tissue of heart 102.

The fixation mesh and anti-rotation functionality described herein are described primarily in the context of a cardiac pacemaker configured to be implanted in one chamber and deliver pacing and sense in that chamber and an additional chamber. However, the anti-rotation features and/or functionality described herein may be included on any implantable medical device, such as an implantable stimulator or implantable lead configured to be fixed at any location or tissue of the body. For example, an implantable stimulator or implantable lead may include one or more tissue retention features on a distal end, the one or more tissue retention features being configured to prevent or inhibit unintended rotation and/or dislodgement of the implantable stimulator or implantable lead from tissue of the patient.

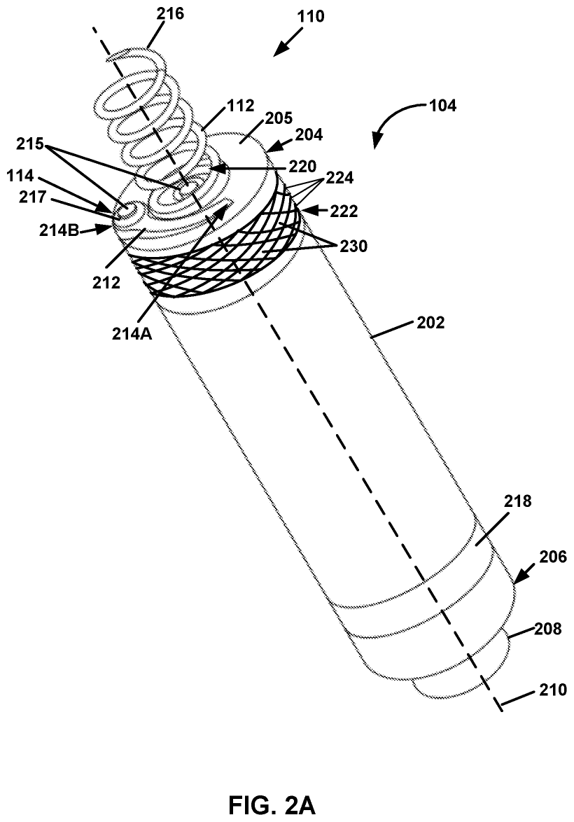

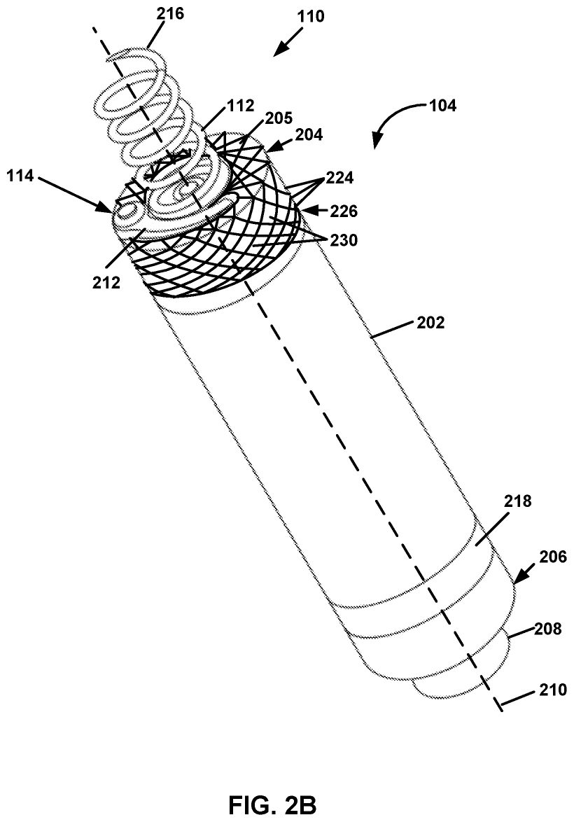

FIG. 2A is a perspective diagram illustrating device 104. Device 104 may include a housing 202 extending from a distal end 204 to a proximal end 206 along longitudinal axis 210. First electrode 112 and second electrode 114 may extend distally along from distal end 204 of housing 202 and along longitudinal axis 210. Device 104 may include a fixation mesh 222 extending at least partially around an outer perimeter of distal end 204 of housing 202. In some examples, as illustrated in FIG. 2A, fixation mesh 222 may not be disposed over distal face 205 of distal end 204 of housing 202.

Housing 202 may define a hermetically sealed internal cavity. Housing 202 may be formed from a conductive material including titanium or titanium alloy, stainless steel, MP35N (a non-magnetic nickel-cobalt-chromium-molybdenum alloy), platinum alloy or other bio-compatible metal or metal alloy, or other suitable conductive material. In some examples, housing 202 is formed from a non-conductive material including ceramic, glass, sapphire, silicone, polyurethane, epoxy, acetyl co-polymer plastics, polyether ether ketone (PEEK), a liquid crystal polymer, other biocompatible polymer, or other suitable non-conductive material.

Housing 202 extends between distal end 204 and proximal end 206 along longitudinal axis 210. Housing 202 may be cylindrical or substantially cylindrical but may be other shapes, e.g., prismatic, or other geometric shapes. Housing 202 may include a delivery tool interface member 208, e.g., at proximal end 206, for engaging with a delivery tool during implantation of device 104. At distal end 204, housing 202 may define a face 205 of housing 202. Face 205 may define a distal end major surface. Face 205 may be orthogonal to longitudinal axis 210. In some examples, face 205 is slanted, e.g., face 205 defines a reference plane that is not orthogonal to longitudinal axis 210.

Face 205 may define a protrusion 212 extending distally from face 205. Second electrode 114 may be disposed on protrusion 212 (e.g., on a distal surface of protrusion 212). Protrusion 212 may separate second electrode 114 from face 205 by a fixed distance, e.g., to improve sensing and/or pacing capabilities of electrode 114. Protrusion 212 may be a ramp extending from face 205, an elongated member protruding from face 205, or any other structure configured to separate second electrode 114 from face 205.

Fixation mesh 222 may be disposed around an outer perimeter of distal end 204 of housing 202. Fixation mesh 222 may include a plurality of interconnecting filaments 224. Filaments 224 may define a plurality of openings 230 within fixation mesh 222. Fixation mesh 222 may allow for growth of tissue through the plurality of openings 230, between fixation mesh 222 and an outer surface of housing 202, and around at least some of filaments 224. For example, at intersections between two or more filaments 224 within fixation mesh 222, the two or more filaments 224 may define space(s) between the outer surface of housing 202 and the two or more filaments 224. Each intersection may be formed from two or more filaments 224. Filaments 224 may extend along fixation mesh 222 in two or more directions. In some examples, as illustrated in FIG. 2A, a first plurality of filaments 224 extend around the outer perimeter of housing 202 in a counterclockwise direction and a second plurality of filaments 224 extend around the outer perimeter of housing 202 in a clockwise direction. Different plurality of filaments 224 may extend along longitudinal axis 210, orthogonal to longitudinal axis 210, in a clockwise direction about longitudinal axis 210 at varying helical pitches, or in a counterclockwise direction about longitudinal axis at varying helical pitches. Tissue may grow into the space(s) and around the two or more filaments 224 to affix device 104 within the tissue. In some examples, one or more therapeutic substances (e.g., an anti-microbial substance, an anti-fungal substance, an anti-bacterial substance, a steroid, an analgesic) may be disposed over one or more of filaments 224 and may reduce inflammation of the tissue at the target implant site.

Each filament 224 may be an elongated member formed from one or more biocompatible materials (e.g., polyethylene, polyethylene terephthalate, glycoprene, polypropylene). A length of each elongated member may be substantially greater (e.g., tens of times or hundreds of times greater) than a width of the elongated member. Each filament 224 may be formed from a single strand of biocompatible material(s) or two or more strands (e.g., two or more woven or twisted-together strands) of biocompatible material(s). Two or more interconnecting filaments 224 may form an opening 230 of the plurality of openings 230 in fixation mesh 222. Each opening 230 may allow ingress of tissue into spaces between fixation mesh 222 and housing 202 and/or spaces around filaments 224.

Filaments 224 may define openings 230 with uniform dimensions (e.g., uniform widths, uniform lengths) or openings 230 with different dimensions across fixation mesh 222. The dimensions of the openings 230 may be dependent on the density of filaments 224 for a given area of fixation mesh 222. The density of filaments 224 may be defined by a picks per inch (p.p.i.) value for the given area. Picks per inch may represent a number of separate filaments 224 extending along a same direction (e.g., extending along a same reference axis) within the given area. A larger p.p.i. value corresponds to a greater density of filaments 224, and vice versa 224. A portion of fixation mesh 222 more distal to distal end 204 of housing 202 may include filaments 224 defining a larger p.p.i. value, e.g., to increase a number of intersection locations between filaments 224 and thereby an available volume for ingrown tissue. The dimensions of filaments 224 and openings 230 may be similar to or the same as the filaments defining at least a portion of TYRX™ Absorbable Antibacterial Envelope of Medtronic Inc., Minneapolis, Minnesota.

Fixation mesh 222 may not extend over face 205 of distal end 204. Device 104 may include one or more features (e.g., protrusion 212, first electrode 112, one or more other electrodes, other anti-rotation features, therapeutic substance dispensing devices 215) on face 205 of housing 202. Fixation mesh 222 may not cover face 205 to prevent impedance of the one or more features by one or more filaments 224 of fixation mesh 222. As illustrated in FIG. 2A, fixation mesh 222 may affix device 104 to tissue surround an outer perimeter of distal end 204 of housing 202 and may not affix device to tissue contacting face 205 of device 104.

Fixation mesh 222 may be affixed to device 104. In some examples, distal end 204 of housing 202 may define a groove defining a reduced-diameter portion around the outer perimeter of housing 202. The groove may be sized to receive fixation mesh 222. For example, the groove may define a height along longitudinal axis 210 greater than or equal to a height of fixation mesh 222 along longitudinal axis 210. The reduction in diameter from a maximum outer diameter of housing 202 to a minimum outer diameter of the groove may be greater than or equal to a thickness of fixation mesh 222. When fixation mesh 222 is disposed within the groove, the fixation mesh 222 may not increase an overall outer diameter of housing 202. Fixation mesh 222 may be retained within the groove via friction, reactive forces acting on fixation mesh 222, an adhesive, or an attachment member. In some examples, fixation mesh 222 may be affixed to device 104 via an adhesive or via an attachment member on housing 202. Filaments 224 defining a distal end or a proximal end of fixation mesh 222 may be coupled to the outer surface of housing 202 via an adhesive or may be coupled to a first attachment member. The first attachment member may be configured to interact with a corresponding second attachment member on housing 202 to secure fixation mesh 222 to housing 202.

Distal end 204 may include one or more protrusions 212. Protrusion 212 may be disposed radially outwards of first electrode 112 relative to longitudinal axis 210. Second electrode 114 may be disposed on protrusion 212. Protrusion 212 may extend from a first end 214A that is fixedly attached to housing 202 at or near distal end 204 (e.g., attached to face 205), to a second end 214B that is more distal than the first end. Second electrode 114 may be disposed on second end 214B. In the example illustrated in FIG. 2A, wherein protrusion 212 is a ramp, the ramp may extend form first end 214A to second end 214B. The ramp may extend around at least a portion of a perimeter of housing 202 and may extend up to 180 degrees around longitudinal axis 210 and along the perimeter of housing 202. The ramp of protrusion 212 may define a partial helix, e.g., wound in a same direction and/or in different directions (e.g., in an opposite direction) around longitudinal axis 210 as a helix and/or coil defined by first electrode 112. In some examples, protrusion 212 defines a partial helix wound in a clockwise direction and first electrode 112 defines a helix wound in a counterclockwise direction. In some examples, protrusion 212 defines a partial helix wound in a counterclockwise direction and first electrode 112 defines a helix wound in a clockwise direction.

First electrode 112 may include one or more coatings (e.g., electrically insulative coating(s)) configured to define a first electrically active region 216, or first electrode 112 may otherwise define first electrically active region 216. In some examples, first electrically active region 216 is more proximate to the second, e.g., distal, end of first electrode 112. In the example of FIG. 2A, first electrically active region 216 includes the distal end of electrode 112. Second electrode 114 may include one or more coatings configured to define a second electrically active region 217 on an outer surface of second electrode 114. In some examples, as illustrated in FIG. 2A, second electrical active region 217 forms a ring around a therapeutic substance dispensing device 215 on second electrode 114. Second electrode 114 may include, but is not limited to, a button electrode, a spring electrode, or any other suitable type or shape of electrode. Second electrically active region 217 may be separated from face 205 by a fixed distance by protrusion 212.

First and second electrodes 112 and 114 may be formed of an electrically conductive material, such as titanium, platinum, iridium, tantalum, stainless steel or alloys thereof. First and second electrodes 112 and 114 may be coated with an electrically insulating coating, e.g., a parylene, polyurethane, silicone, epoxy, or other insulating coating, to reduce the electrically conductive active surface area of first and second electrodes 112 and 114, and thereby define first and second electrically active regions 216 and 217. Defining first and second electrically active regions 216 and 217 by covering portions with an insulating coating may increase the electrical impedance of first and second electrodes 112 and 114 and thereby reduce the current delivered during a pacing pulse that captures the cardiac tissue. A lower current drain conserves the power source, e.g., one or more rechargeable or non-rechargeable batteries, of device 104.

In some examples, first and second electrodes 112 and 114 include an electrically conducting material coating on first and second electrically active regions 216 and 217 to define the active regions. For example, first and second electrically active regions 216 and 217 may be coated with titanium nitride (TiN). First and second electrodes 112 and 114 may be made of substantially similar material or may be made of different material from one another.

In the example of FIG. 2A, first electrode 112 takes the form of a helix or a coil. First electrode 112 may be an elongated body defining a helix. In some examples, a helix is an object having a three-dimensional shape like that of a wire wound uniformly in a single layer around a cylindrical or conical surface or mandrel such that the wire would be in a straight line if the surface were unrolled into a plane. First electrode 112 may extend from face 205 from a proximal end 220 to a distal end, e.g., defining first electrically active region 216. Proximal end 220 may be a location along first electrode 112 where first electrode 112 extends distally past distal end 204 of device 104.

Second electrode 114 is disposed on distal end 204 and may include a button electrode, e.g., as illustrated in FIG. 2A, or any other suitable type or shape of electrode. In some examples, device 104 includes a plurality of second electrodes 114 (e.g., two or more second electrodes 114) disposed on distal end 204 of housing 202. The plurality of second electrodes 114 may be equally spaced around a circumference of distal end 204. At least one of the plurality of second electrodes 114 may be disposed protrusions 212. In some examples, each of the plurality of second electrodes 114 is disposed on a different protrusion 212. Each protrusion 212 may include a single second electrode 114 or two or more second electrodes 114. In some examples, second electrode 114 is disposed at a predetermined angle, e.g., about longitudinal axis 210, away from proximal end 220 of first electrode 112.

In some examples, first electrode 112 includes one or more anti-rotation features in addition to fixation mesh 222. The additional anti-rotation features may facilitate fixation of distal end 205 of device 104 to the tissue. The additional anti-rotation features may include a shape of first electrode 112, dimensions (e.g., outer diameter, pitch, or the like) of first electrode 112, one or more features disposed on an outer surface of first electrode 112, or the like. The shape and/or dimensions of first electrode 112 may include a geometric shape of first electrode 112, a varying diameter configuration of first electrode 112, a varying pitch configuration of first electrode 112, a waveform configuration of first electrode 112, or any combination herein. The one or more anti-rotation features disposed on first electrode 112 may include, but are not limited to, elongate darts, barbs, or tines. In some examples, the anti-rotation features include bumps, ridges, and/or other texturing disposed on one or more surfaces of ramp 214 and/or of face 205. The one or more anti-rotation features may resist rotation of first electrode 112 (e.g., by penetrating the tissue, by increasing the friction between first electrode 112 and the tissue, or the like) alone or in conjunction with protrusions 212 and/or with fixation mesh 222.

As illustrated in FIG. 2A, first electrode 112 may be a helix extending distally from face 205 at proximal end 220 and revolving around longitudinal axis 210 in a counter-clockwise direction (i.e., “wound” in a counter-clockwise direction, and ramp 214 may define partial helix extending distally from face 205 and revolving around longitudinal axis 210 in a clockwise direction, although in other examples the first electrode 112 and ramp 214 may revolve around longitudinal axis 210 in different directions (e.g., first electrode 112 revolves around longitudinal axis 210 in a clockwise direction and ramp 214 revolves around longitudinal axis 210 in a counter-clockwise direction) or first electrode 112 and ramp 214 may revolve around longitudinal axis 210 in a same direction.

First and second electrodes 112 and 114 may vary in size and shape in order to enhance tissue contact of first and second electrically active regions 216 and 217. For example, first electrodes 112 may have a round cross-section or could be made with a flatter cross-section (e.g., oval or rectangular) based on tissue contact specifications. In some examples, second electrode 114 defines an outer surface that varies in size and shape (e.g., an oval outer surface, an outer surface with a larger diameter, or the like) in order to enhance tissue contact of second electrically active region 217.

The size and shape of first electrode 112 may be determined at least in part by stiffness requirements. For example, stiffness requirements may vary based on the expected implantation requirements, including the tissue into which the electrodes are implanted or contact, as well as how long device 104 is intended to be implanted.

The distal end of first electrode 112 can have a conical, hemi-spherical, or slanted edge distal tip with a narrow tip diameter, e.g., less than 1 millimeter (mm), for penetrating into and through tissue layers. The distal end of first electrode can be a sharpened or angular tip or sharpened or beveled edges, but the degree of sharpness may be constrained to avoid a cutting action that could lead to lateral displacement of the distal end of first electrode 112 and undesired tissue trauma. In some examples, first electrode 112 defines a maximum diameter at its base that interfaces with housing distal end 204. In such examples, the outer diameter of the helix defined by first electrode 112 may decrease from housing distal end 204 to the distal end of first electrode 112. In some examples, the diameter of first electrode 112 varies from proximal end 220 to the distal end of first electrode 112. The varying diameter may cause first electrode 112 to resist rotation within the tissue of heart 102.

The outer dimensions of first electrode 112 can be substantially straight and cylindrical, with first electrode 112 being rigid in some examples. First electrode 112 may have flexibility in lateral directions, being non-rigid to allow some flexing with heart motion. In a relaxed state, when not subjected to any external forces, first electrode 112 can be configured to maintain a distance between first electrically active region 216 and housing distal end 204.

Distal end of first electrode 112 can pierce through one or more tissue layers to position first electrically active region 216 within a desired tissue layer, e.g., the ventricular myocardium 108 or interventricular septum. Accordingly, first electrode 112 extends a distance from housing distal end 204 corresponding to the expected pacing site depth and may have a relatively high compressive strength along its longitudinal axis, which may be substantially similar to or coincident with longitudinal axis 210, to resist bending in a lateral or radial direction when a longitudinal, axial, and/or rotational force is applied, e.g., to the proximal end 206 of housing 202 to advance device 104 into the tissue at target implant region 106. By resisting bending in a lateral or radial direction, first electrode 112 can maintain a spacing between a plurality of windings of first electrode 112 when first electrode 112 is a helix electrode. The spacing may be a pre-determined pitch of first electrode 112 and may vary from distal end 204 to the distal end of first electrode 112. First electrode 112 may be longitudinally non-compressible. First electrode 112 may also be elastically deformable in lateral or radial directions when subjected to lateral or radial forces, however, to allow temporary flexing, e.g., with tissue motion, but returns to its normally straight position when lateral forces diminish. In some examples, when first electrode 112 is not exposed to any external force, or to only a force along its longitudinal axis (substantially similar to or coincident with longitudinal axis 210), first electrode 112 retains a straight, linear configuration as shown.

All, substantially all, or a portion of housing 202 may function as an electrode 218, e.g., an anode, during pacing and/or sensing. In some examples, electrode 218 circumscribes a portion of housing 202 at or near proximal end 206. Electrode 218 can fully or partially circumscribe housing 202. FIG. 2A shows electrode 218 extending as a singular band around the outer perimeter of housing 202. Electrode 218 can also include multiple segments spaced a distance apart along a longitudinal axis 210 of housing 202 and/or around a perimeter of housing 202. In some examples, electrode 218 is disposed on face 205 or on ramp 214 disposed on face 205. In some examples, second electrode 114 is disposed on a first ramp 214 and electrode 218 may be disposed on a second ramp 214.

When housing 202 is formed from a conductive material, such as a titanium alloy, portions of housing 202 may be electrically insulated by a non-conductive material, such as a coating of parylene, polyurethane, silicone, epoxy or other biocompatible polymer, or other suitable material. For the portions of housing 202 without the non-conductive material, one or more discrete areas of housing 202 with conductive material can be exposed to define electrode 218.

When housing 202 is formed from a non-conductive material, such as a ceramic, glass or polymer material, an electrically-conductive coating or layer, such as a titanium, platinum, stainless steel, alloys thereof, a conductive material may be applied to one or more discrete areas of housing 202 to form electrode 218.

In some examples, electrode 218 is a component, such as a ring electrode, that is mounted or assembled onto housing 202. Electrode 218 may be electrically coupled to internal circuitry of device 104 via electrically-conductive housing 202 or an electrical conductor when housing 202 is a non-conductive material. In some examples, electrode 218 is located proximate to proximal end 206 of housing 202 and can be referred to as a proximal housing-based electrode. Electrode 218 can also be located at other positions along housing 202, e.g., located proximately to distal end 204 or at other positions along longitudinal axis 210.

In some examples, second electrode 114 or electrode 218 is paired with first electrode 112 for sensing ventricular signals and delivering ventricular pacing pulses. In some examples, second electrode 114 is paired with electrode 218 or first electrode 112 for sensing atrial signals and delivering pacing pulses to atrial tissue (e.g., to the atrial endocardium) in target implant region 106. In other words, electrode 218 is paired, at different times, with first electrode 112 and/or second electrode 114 for either ventricular or atrial functionality, respectively. In some examples, first and second electrodes 112 and 114 are paired with each other, with different polarities, for atrial and ventricular functionality.

In some examples, second electrode 114 is configured as an atrial cathode electrode for delivering pacing pulses to the atrial tissue, e.g., at target implant region 106 in combination with electrode 218. Second electrode 114 and electrode 218 may also be used to sense atrial P-waves for use in controlling atrial pacing pulses (delivered in the absence of a sensed P-wave) and for controlling atrial-synchronized ventricular pacing pulses delivered using first electrode 112 as a cathode and electrode 218 as the return anode.

A distal end of first electrode 112 can be configured to rest within a ventricular myocardium of the patient, and second electrode 114 can be configured to contact an atrial endocardium of the patient without penetration of the atrial endocardium. Device 104 may include more or fewer electrodes than two electrodes. In some examples, device 104 includes one or more second electrodes 114 along housing distal end 204. For example, device 104 may include two or three electrodes configured for atrial functionality like second electrode 114, and the three electrodes may be substantially similar or different from one another. Spacing between a plurality of second electrodes 114 may be at an equal or unequal distance. Second electrode(s) 114 may be individually selectively coupled to sensing and/or pacing circuitry enclosed by housing 202 for use as an anode with first electrode 112 or as an atrial cathode electrode, or may be electrically common and not individually selectable. In some examples, in place of first electrode 112, device 104 includes a fixation element (not shown) of similar shape and mechanical, but without an electrically active region or electrode formed thereon or borne thereby; in such examples, electrically active region 216 can be positioned on a separate member and/or on the housing 202. In some examples, device 104 only includes first electrode 112 and electrode 218 and does not include any second electrodes 114.

Inflammation of patient tissue may result from interaction with device 104. For example, penetration of tissue by first electrode 112 and/or contact between tissue and second electrode 114 may result in inflammation of the tissue. In some examples, tissue ingrowth into fixation mesh 222 leads to inflammation of the tissue. Inflammation of patient tissue proximate to first and second electrodes 112 and 114 may result in higher thresholds for stimulation delivered to the tissue to activate, or capture, the tissue. Higher capture thresholds may, in turn, increase the consumption of a power source of device 104 associated with delivery of the stimulation.

In some examples device 104 includes one or more therapeutic substance dispensing devices 215, e.g., on face 205, within a recess defined by second electrode 114. Therapeutic substance dispensing devices 215 may be configured to elute one or more steroids to tissue in proximity to therapeutic substance dispensing devices 215 over time. The steroid may mitigate inflammation of patient tissue resulting from interaction with the IMD. In some examples, therapeutic substance dispensing devices 215 comprise one or more monolithic controlled release devices (MCRDs). In some examples, the therapeutic substance is disposed on filaments 224 instead of or in addition to therapeutic substance dispensing devices 215.

FIG. 2B is a perspective diagram illustrating another example configuration of the device 104 of FIG. 1 with a fixation mesh 226 disposed over distal end 204 of device 104. As illustrated in FIG. 2B, fixation mesh 226 may be disposed over distal face 205. Disposal of fixation mesh 226 over distal face 205 may facilitate ingrowth of tissue around portions of fixation mesh 226 covering face 205, thereby increasing a surface area of fixation and the strength of the bond between device 104 and the tissue of heart 102.

Face 205 may include one or more features protruding from face 205 or extending from distal end 204. The one or more features may include, but are not limited to, first electrode 112, second electrode 114, protrusions 212, therapeutic substance dispensing devices 215, other electrodes, or other anti-rotation or fixation features. Filaments 224 of fixation mesh 226 may define one or more openings in fixation mesh 226 sized to receive corresponding features on face 205 of device 104. A manufacturer may form the one or more openings as a part of manufacturing fixation mesh 226. A manufacturer may form the one or more openings by determining the placement and dimensions of the one or more features and removing material (e.g., via a cutting element) from a pre-formed fixation mesh 226 to define the one or more openings in accordance with the placement and dimensions of the one or more features.

Each feature on face 205 may extend through a corresponding opening in fixation mesh 226. The openings in fixation mesh 226 may allow the one or more features to interact with tissue of heart 102 (e.g., to penetrate the tissue, to contact the tissue) without intervention from filaments 224 of fixation mesh 226.

In some examples, instead of the fixation meshes illustrated in FIGS. 2A and 2B, an example fixation mesh may only be disposed over face 205 of housing 202. In such examples, the ingrown tissue affixes face 205 of device 104 to the tissue. The example fixation mesh may be affixed to face 205 and/or to distal end 204 of device 104 via an adhesive or attachment mechanism, e.g., as previously described herein.

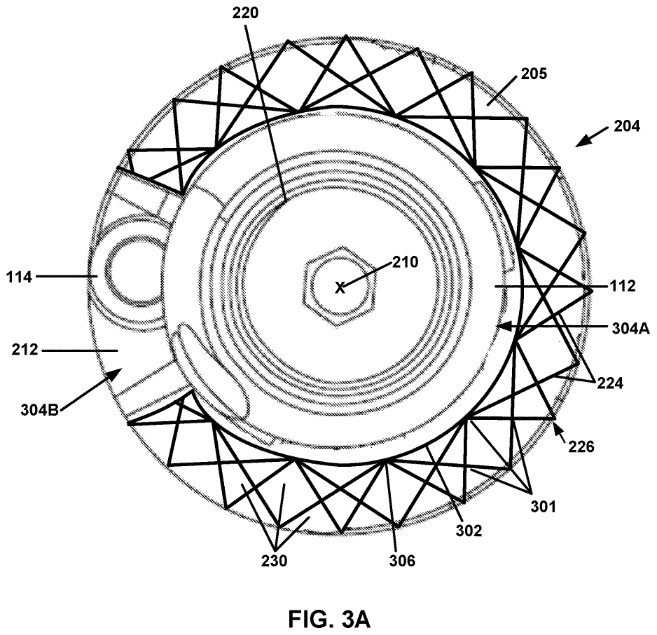

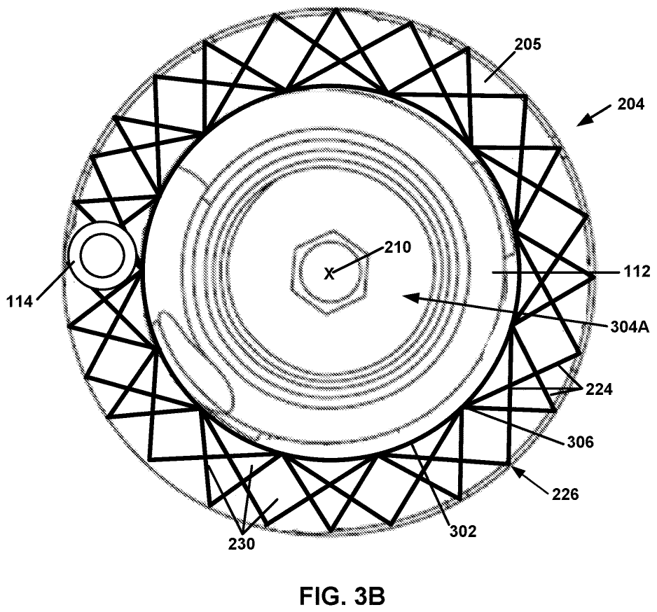

FIG. 3A is a perspective diagram illustrating a top-down view of an example configuration of device 104 of FIG. 2B. As illustrated in FIG. 3A, fixation mesh 226 may be disposed over face 205 and filaments 224 of fixation mesh 226 may define openings 304A, 304B (collectively referred to herein as “openings 304”). Each opening 304 may be configured to retain a feature extending from face 205 of device 104, including, but are not limited to, first electrode 112, protrusion 212 (illustrated in FIG. 3A as a ramp), and second electrode 114. While the example fixation mesh 226 illustrated in FIG. 3A defines two openings 304, other example fixation meshes may include one opening 304 or three or more openings 304. Each opening 304 may correspond to a specific feature on face 205. For example, opening 304A corresponds to first electrode 112 and opening 304B corresponds to protrusion 212 and second electrode 114. Each opening 304 may only be configured to be disposed around the corresponding feature. In some examples, each opening 304 is configured to by disposed around any feature on face 205.

Fixation mesh 226 may include one or more filaments 302 connecting to filaments 224. Filaments 302 may define an outer perimeter of each of openings 304. Filaments 302 may define minimum dimensions for each opening 304 (e.g., a minimum width, a minimum length) such that each feature on face 205 may extend through a corresponding opening 304. Filaments 302 may be of the same or different dimensions than filaments 226. Filaments 302 may be formed from a same number and/or type of strands as filaments 226 or may be formed from a different number and/or types of strands as filaments 226. Filaments 226 may intersect with filaments 302 at locations 306 along filaments 302. At each location 306, filaments 226 may be wrapped around, adhered to, or otherwise secured to filament 302 and one or more other filaments 226. For example, as illustrated in FIG. 3A, at each location 306, up to four separate filaments 226 are affixed to filament 302. The placement of locations 302 along filament 302 and/or the number of filaments 226 at each location 302 may facilitate the formation of specific patterns of openings 230 formed by interconnecting struts 226 on face 205 and/or around the outer perimeter of housing 202, as illustrated in FIGS. 2A and 2B.

In some examples, fixation mesh 226 may not include one or more filaments 302. A manufacturer may remove material from fixation mesh 226 (e.g., remove portions of at least one filament 224) to form openings 304 in fixation mesh 226. The manufacturer may align openings 304 with features on face 205 and place fixation mesh 226 over face 205 such that each feature extends through a corresponding opening 304. The manufacturer may remove material from fixation mesh 226 via a cutting instrument (e.g., a laser-cutting instrument, an etching instrument, a bladed instrument, an abrasion instrument). In some examples, the manufacturer applies thermal energy to one or more filaments 224 to remove material from fixation mesh 226 or to fuse filaments 224 at a plurality of locations around each opening 304 (e.g., to define an outer perimeter of each opening 304).

FIG. 3B is a perspective diagram illustrating a top-down view of another example configuration of device 104 of FIG. 2B. Some devices 104 may not include protrusions 212 as illustrated in FIG. 3A. Fixation mesh 226 may define a single opening 304A may not include any other openings 304 for other features on face 205. Second electrode 114 and other features may be disposed within openings 230 formed by interconnecting filaments 224, 302. A manufacturer may dispose fixation mesh 226 over face 205 and then dispose features (e.g., second electrode 114) onto face 205 through the openings 230. In some examples, a feedthrough assembly of second electrode 114 may extend through openings 230 and into housing 202 to connect second electrode 114 to computing circuitry within housing 202. A main body of second electrode 114 may define a larger outer diameter than the feedthrough assembly of second electrode 114 and may be disposed on top of fixation mesh 226. The fixation mesh 226 and the design of second electrode 114 may separate second electrically conductive active region 217 on second electrode 114 from face 205 by the fixed distance.

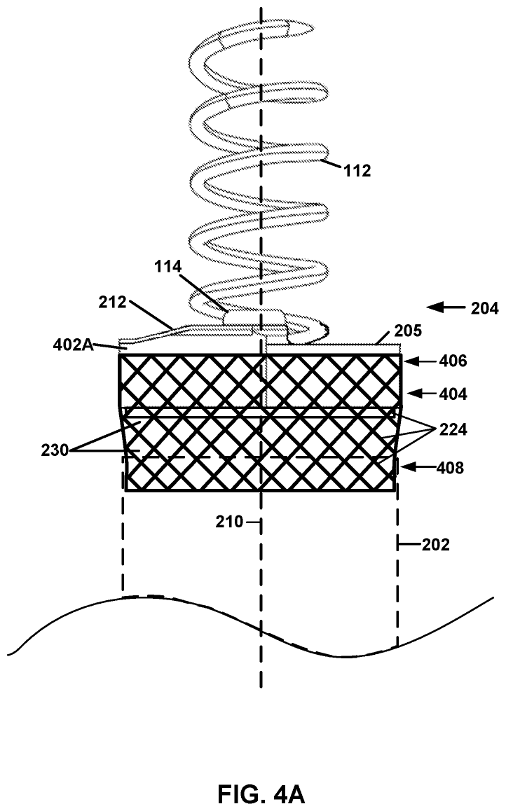

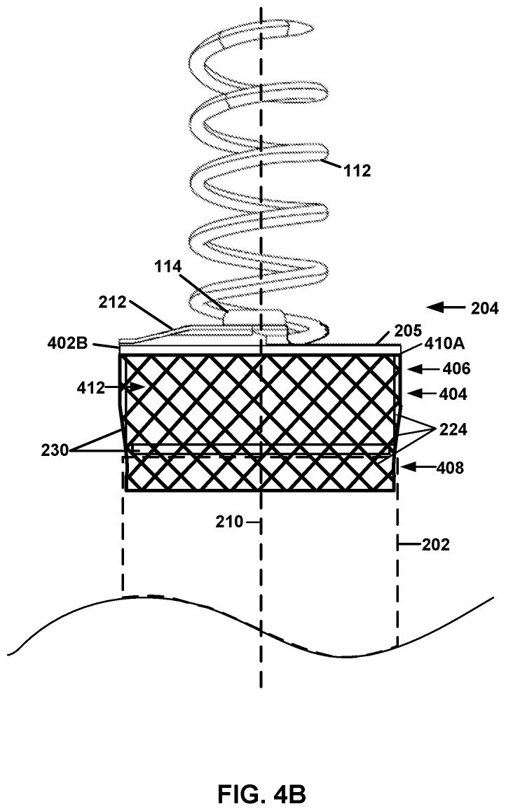

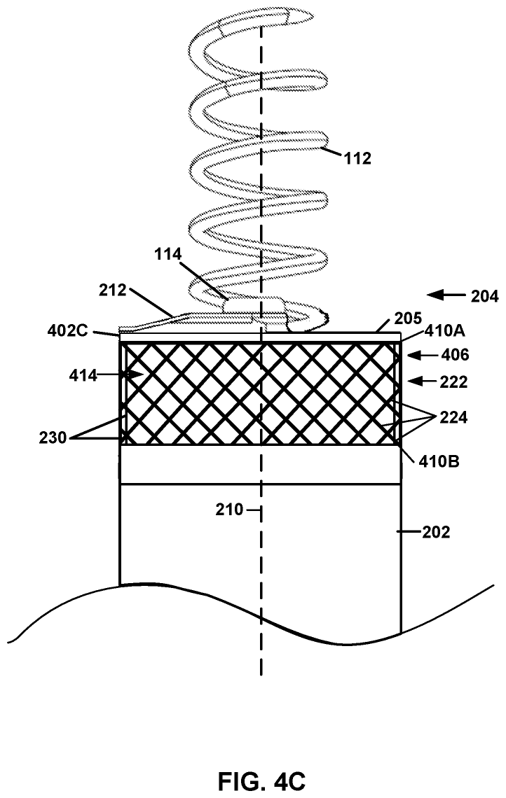

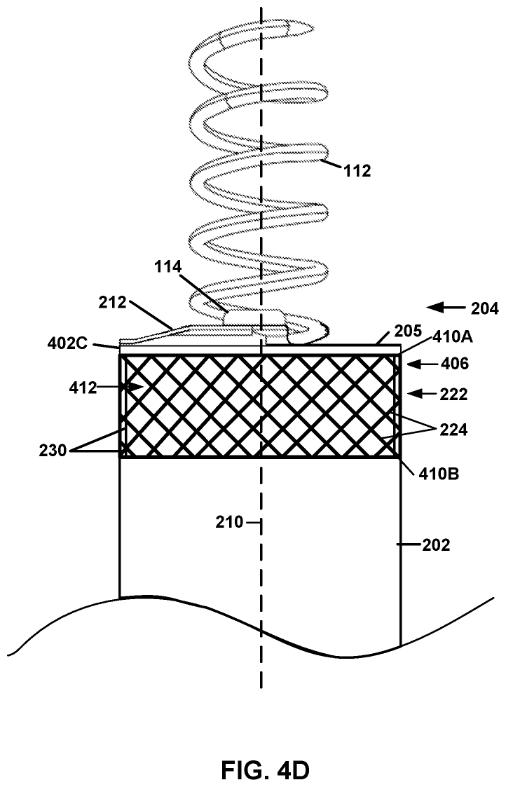

FIG. 4A is a perspective diagram illustrating a side view of a fixation mesh 404 disposed around a removable distal portion 402A of the device 104 of FIG. 2A. In some examples, distal portion 402A (alternative referred to herein as “header 402A”) may define distal end 204 and face 205 of device 104. Electrodes 112, 114 and protrusions 212 may be disposed on or coupled to header 402A. Header 402A and housing 202 may define an inner volume of housing 202. Internal circuitry of device 104 may be disposed within the inner volume. Feedthrough assemblies for electrodes 112, 114 may extend proximally from header 402A and into the inner volume. The feedthrough assemblies may be coupled to the internal circuitry of device 104.

Header 402A may be removable from housing 202. Removable header 402A may simplify manufacture of device 104 and/or may facilitate adjustments or replacement of fixation features (e.g., first electrode 112) of device 104 without altering the internal circuitry of device 104. Header 402A may be removed from housing 202 via application of tensile force to header 402A and/or by rotating header 402A about longitudinal axis 210 and relative to housing 202.

Fixation mesh 404 may be formed by a plurality of filaments 224. Fixation mesh 404 may extend along longitudinal axis 210 from a distal end 406 to a proximal end 408. Fixation mesh 404 may be similar to or the same as fixation meshes 222, 226, in terms of function and dimensions. Fixation mesh 404 may be disposed around the outer perimeter of header 402A, as illustrated in FIG. 4A, and/or may be disposed over face 205 in a manner similar to fixation mesh 226 of FIGS. 2B-3B.

Proximal end 408 of fixation mesh 404 may extend proximally past a proximal end of header 402A. A portion of fixation mesh 404 proximal to the proximal end of header 402A may be configured to be retained within the inner volume of housing 202. When header 402 is affixed to housing 202, fixation mesh 404 may be affixed to device 104 via a compressive force between header 402A and housing 202. When header 402A is affixed to housing 202, fixation mesh 404 may be secured between header 402A and housing 202. The placement of portions of fixation mesh 404 into the inner volume of housing 202 may allow fixation of fixation mesh 404 to device 104 without the use of adhesives and/or other attachment members. Fixation mesh 404 may be released from device 104 via separation of header 402A from housing 202.

FIG. 4B is a perspective diagram illustrating another example side view of a fixation mesh 404 disposed around a removable distal portion 402B of the device of FIG. 2A. Removable distal portion 402B (also referred to herein as “header 402B”) may be of similar structure and dimensions to header 402A and may be secured to or removed from housing 202 in a similar manner. Header 402B may define a shoulder 410A extending around an outer perimeter of header 402B and separating distal end 204 of header 402B from a groove 412 around the outer perimeter of header 402B. Groove 412 may define a reduced outer diameter relative to distal end 204 and may be configured to retain fixation mesh 404 around the outer surface of header 402B and within groove 412.

Shoulder 410A may define a depth from an outer perimeter of distal end 204 to an outer surface of groove 412 along a reference plane orthogonal to longitudinal axis 210 greater than or equal to a thickness of fixation mesh 404 along the reference plane, e.g., to facilitate disposal of fixation mesh 404 around header 402B without increasing a maximum outer diameter of header 402B. Shoulder 410A may inhibit unintended movement of fixation mesh 404 towards distal end 204 of header 402B.

FIGS. 4C and 4D are perspective diagrams illustrating other example side views of a fixation mesh (e.g., fixation mesh 222) disposed around a distal end 204 of device 104 of FIG. 2A. distal portions 402C, D (alternative referred to herein as “header 402C,” “header 402D”) may define distal end 204 and face 205 of device 104. Headers 402C, 402D may be manufactured separately from housing 202 and may be removably or permanently affixed to housing 202. In some examples, as illustrated in FIG. 4C, header 402C defines a first shoulder 410A and a second shoulder 410B proximal to first shoulder 410A. In some examples, as illustrated in FIG. 4D, header 402D defines one shoulder 410A and a distal end of housing 202 defines a second shoulder 410B, Second shoulder 410B may define a same or different (e.g., greater) outer diameter than first shoulder 410A, or another portion of headers 402C, 402D or of housing 202. Shoulders 410A, 410B may define groove 414. Groove 414 may extend around an entirety of the outer perimeter of header 402C or of header 402D. Groove 414 may define a width along longitudinal axis 210 greater than or equals to the width of fixation mesh 222 along longitudinal axis 210, e.g., such that groove 414 retains an entirety of fixation mesh 222.

Shoulders 410A, 410B may define a same or different depth along a reference plane orthogonal to longitudinal axis 210 from the outer perimeter of distal end 204 to the outer surface of groove 414. Shoulders 410A, 410B may define depths greater than or equals to a thickness of fixation mesh 222 along the reference plane, e.g., such that fixation mesh 222 may be disposed within groove 414 without increasing a maximum outer diameter of device 104. Shoulders 410A, 410B may inhibit unintended movement of fixation mesh 222 along longitudinal axis 210. For example, shoulder 410A inhibits distal movement of fixation mesh 222 along longitudinal axis 210 and shoulder 410B inhibits unintended movement of fixation mesh 222 along longitudinal axis 210.

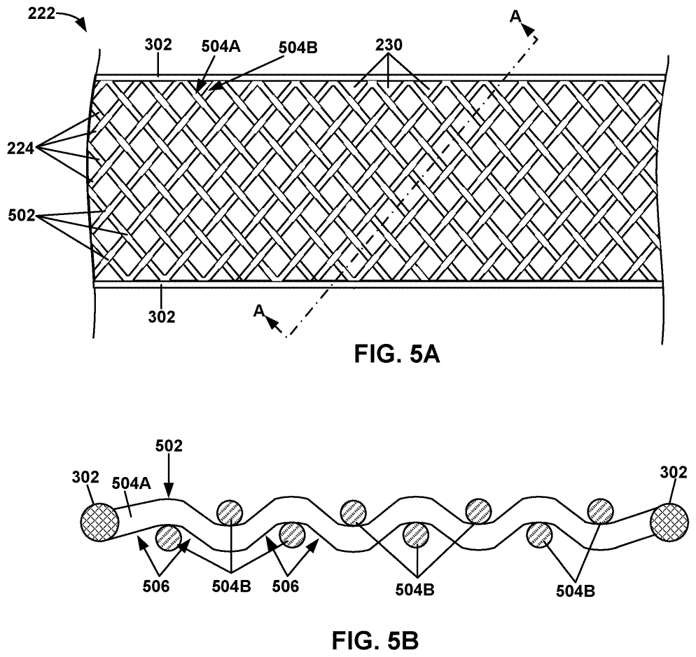

FIG. 5A is a perspective diagram illustrating an example configuration of a fixation mesh 222. FIG. 5B is a cross-section diagram illustrating a cross-section view of the example configuration of the fixation mesh 222 of FIG. 5A, the cross-section being take along line A-A of FIG. 5A and along the length of a filament 224 of fixation mesh 222. While FIGS. 5A and 5B primarily describe fixation mesh 222, other fixation meshes described herein (e.g., fixation mesh 226, fixation mesh 404) may include similar filaments 224 having the same or similar dimensions and defining same or similar openings 230 within the mesh.

Filaments 224 may include filaments 504A extending in a first direction and filaments 504B extending in a second direction. In some examples, as illustrated in FIG. 5A, filaments 504A, 504B (collectively referred to herein as “filaments 504”) extend from one end of fixation mesh 222 to an opposite end of fixation mesh 222 in two separate directions. In some examples, filaments 504 may extend between the ends of fixation mesh 222 in three or more directions. Filaments 504 extending in different directions may be separated by angles of up to 90 degrees. For example, as illustrated in FIG. 5A, filaments 504A are separated from filaments 504B by an angle up to 90 degrees. Filaments 504 may define uniform or different dimensions. For example, filaments 504A may define the same or different dimensions than filaments 504B.

Filaments 504 may intersect at locations 502 across fixation mesh 222. At each location 502, one filament 504 may cross over and be disposed over one or more other filaments 504. For example, as illustrated in FIG. 5B, at each location 502, a filament 504A may be disposed radially outside of a filament 504B, or vice versa. Filaments 504, 504B may be alternatively disposed at a radially external position at locations 502, e.g., to define an interwoven structure along fixation mesh 222. For example, as illustrated in FIG. 5B, filament 504A is disposed over and under alternating filaments 504B.

At each location 502, the overlap between filaments 504 may form spaces 506. When the fixation mesh 222 is affixed to device 104, each space 506 may be defined by filaments 504 and an outer surface of device 104 (e.g., an outer surface of housing 202 or an outer surface of face 205). Tissue of heart 102 may grow into spaces 506 and around filaments 504 to affix fixation mesh 222 and an attached device 104 to the tissue. Spaces 506 may be interconnected and linked via openings 230.

Filaments 302 may define ends of Fixation mesh 222. When fixation mesh 222 is affixed to device 104, filaments 302 may define proximal and distal ends of fixation mesh 222 along longitudinal axis 210 and/or an outer perimeter of openings 304 within fixation mesh 222. Filaments 302 may define a uniform or varying thickness. Filaments 302 may define a same thickness as or different thicknesses than filaments 504. Filaments 504 may be affixed to filaments 302 via an adhesive, or via the wrapping of an end of filament 504 around the outer perimeter of filament 302. Each filament 504 may be affixed to a different filament 302 at each end or may be affixed to a same filament 302 at each end.

Filaments 224 (e.g., filaments 504, 302) may be coated with a therapeutic substance. The therapeutic substance may include, but are not limited to, an anti-bacterial substance, an anti-fungal substance, an anti-microbial substance, a steroid, or an analgesic. The therapeutic substance may reduce inflammation of the tissue of heart 102 in response to fixation mesh 222 any may inhibit the introduction of unintended foreign elements (e.g., foreign microbes, foreign bacteria) to heart 102. The type and concentration of the therapeutic substance(s) may be uniform across fixation mesh 222 or may vary based on the location on fixation mesh 222. For example, portions of fixation mesh 222 configured to be disposed over a face 205 of device 104 may include coatings of different types and/or concentrations of therapeutic substance(s) than a second portion of fixation mesh 222 configured to be disposed around an outer perimeter of housing 202. In some examples, the therapeutic substance may be disposed within openings 230 and cause fixation mesh 222 to define a smooth outer surface. The smooth outer surface may facilitate implantation of device 104 and/or may reduce a risk of introducing a foreign element into the patient. Over time, the tissue of heart 102 may absorb the therapeutic substance within openings 230 and reveal openings 230 in fixation mesh 222.

Filaments 224 may be formed from an absorbable or a non-absorbable material. When filaments 224 are formed from an absorbable material, the tissue of heart 102 may fully absorb the fixation mesh 222 within a period of time (e.g., within a number of weeks, months). When filaments 224 are formed from a non-absorbable material, the fixation mesh 222 may remain over the period of time and may allow for chronic fixation of device 104 to the tissue.

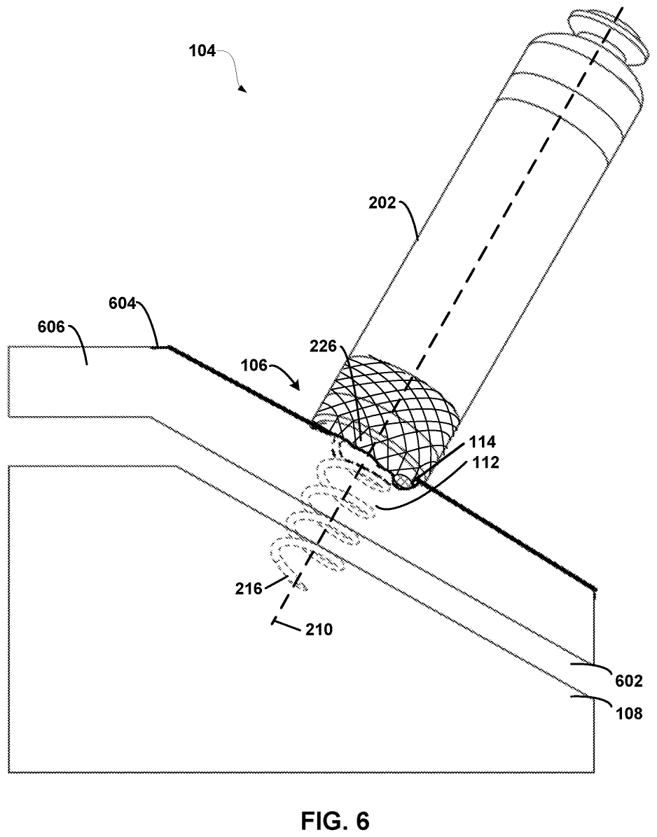

FIG. 6 is a conceptual diagram of the device 104 of FIGS. 1-5B implanted at a target implant site. First electrode 112 may be inserted (e.g., in a manner similar to rotating and advancing a threaded screw) such that tissue becomes engaged with the helix of first electrode 112. As first electrode 112 becomes engaged with tissue, first electrode 112 pierces into the tissue at target implant region 106 and advances through atrial myocardium 606 and central fibrous body 602 to position first electrically active region 216 in ventricular myocardium 108 as shown in FIG. 6. In some examples, first electrode 112 penetrates into the interventricular septum. In some examples, first electrode 112 does not perforate either of the ventricular endocardial or epicardial surface.

In some examples, manual pressure applied to the housing proximal end 206, e.g., via an advancement tool, provides the longitudinal force to pierce the cardiac tissue at target implant region 106. In some examples, actuation of an advancement tool rotates device 104 and first electrode 112 configured as a helix about longitudinal axis 210. The rotation of the helix about the longitudinal axis 210 advances first electrode 112 through atrial myocardium 606 and central fibrous body 602 to position first electrically active region 216 in ventricular myocardium 108 as shown in FIG. 6.

As first electrode 112 advances into the tissue, the distance between second electrode 114 and atrial endocardium 604 decreases until second electrode 114 contact, and may press against, the surface of atrial endocardium 604. Second electrode 114 may press against the surface of atrial endocardium 604 and compress the wall tissue. The compression of the wall tissue may prevent or inhibit rotation of device 104 due to movement of tissue of heart 102 (e.g., movement of ventricular myocardium 108, atrial myocardium 606, central fibrous body 602, or the like) or blood flow during cardiac function. A fixation mesh (e.g., fixation mesh 226) disposed at a distal end 204 may interface with wall tissue of atrial endocardium 604 and facilitate growth of the wall tissue into fixation mesh 226 (e.g., within spaces 506, around filaments 224, within openings 230 of fixation mesh 226). Second electrode 114 is held in contact with atrial endocardium 604 by first electrode 112 by fixation mesh 226. Retraction of second electrode 114 from the surface of atrial endocardium 604 may be prevented or inhibited by first electrode 112 the interaction between the ingrown wall tissue and fixation mesh 226.

The fixation mesh may be the only anti-rotation feature of device 104 in some examples. In some examples, device 104 includes one or more additional anti-rotation features, e.g., defined and/or disposed on first electrode 112, on face 205, on an outer surface of housing 202. The distance by which first electrode 112 extends from housing 202 can be selected so first electrically active region 216 reaches an appropriate depth in the tissue layers to reach the targeted pacing and sensing site, in this case in ventricular myocardium 108, without puncturing all the way through into an adjacent cardiac chamber.

Target implant region 106 in some pacing applications is along atrial endocardium 604, substantially inferior to the AV node and bundle of His. First electrode 112 can have a length that penetrates through atrial endocardium 604 in target implant region 106, through the central fibrous body 602 and into ventricular myocardium 108 without perforating through the ventricular endocardial surface. In some examples, when the full length of first electrode 112 is fully advanced into target implant region 106, first electrically active region 216 rests within ventricular myocardium 108 and second electrode 114 is positioned in intimate contact with atrial endocardium 604. First electrode 112 may extend from housing distal end 204 approximately 3 mm to 12 mm in various examples. In some examples, first electrode 112 may extend a distance from distal end 204 by at least 3 mm, at least 3 mm but less than 20 mm, less than 15 mm, less than 10 mm, or less than 8 mm in various examples. The diameter of an elongated body defining first electrode 112 may be 2 mm or less, e.g., may be 1 mm or less, may be 0.6 mm or less. An outer diameter of the helix or coil defined by first electrode 112 may be 4 mm or less.

As described above and illustrated in FIGS. 2A-6, fixation meshes (e.g., fixation mesh 222, 226, 404) may be defined by a plurality of filaments (e.g., filaments 224, 504). Fixation meshes 222, 226, 404 may be a type of mesh such as, but are not limited to, woven meshes, braided meshes, knitted meshes, or felt meshes. The type of each fixation mesh 222, 226, 404 may be dependent on the dimensions and arrangement of filaments 224, 504 defining the fixation mesh 222, 226, 404.

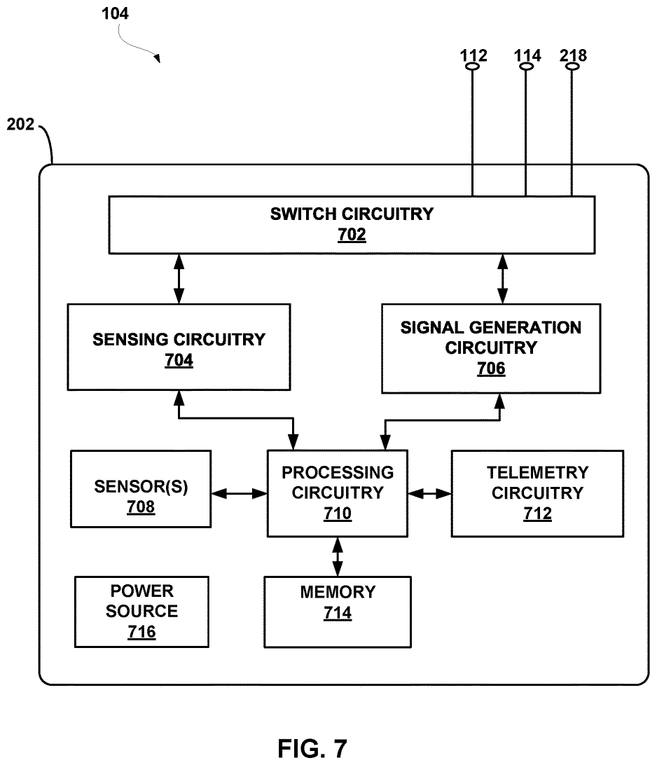

FIG. 7 is a functional block diagram illustrating an example configuration of device 104. As illustrated in FIG. 7, device 104 include electrodes 112 and 114, which may be configured as described with respect to FIGS. 1-6. For example, as described previously, first electrode 112 may be configured to extend from distal end 204 of housing 202 and may penetrate through the wall tissue of a first chamber (e.g., the RA) into wall tissue of a second chamber (e.g., the LV). Second electrode 114 extends from distal end 204 of housing 202 and may be configured to maintain contact with the wall tissue of the first chamber without penetration of the wall tissue of the first chamber by second electrode 114.

In the example shown in FIG. 7, device 104 includes switch circuitry 702, sensing circuitry 704, signal generation circuitry 706, sensor(s) 708, processing circuitry 710, telemetry circuitry 712, memory 714, and power source 716. The various circuitry may be, or include, programmable or fixed function circuitry configured to perform the functions attributed to respective circuitry. Memory 714 may store computer-readable instructions that, when executed by processing circuitry 710, cause device 104 to perform various functions. Memory 714 may be a storage device or other non-transitory medium. The components of device 104 illustrated in FIG. 7 may be housed within housing 202.

Signal generation circuitry 706 generates electrical stimulation signals, e.g., cardiac pacing pulses. Switch circuitry 702 is coupled to electrodes 112, 114, and 218 and may include one or more switch arrays, one or more multiplexers, one or more switches (e.g., a switch matrix or other collection of switches), one or more transistors, or other electrical circuitry. Switch circuitry 702 is configured to direct stimulation signals from signal generation circuitry 706 to a selected combination of electrodes 112, 114, and 218, having selected polarities, e.g., to selectively deliver pacing pulses to the RA, ventricles, or interventricular septum of heart 102. For example, in order to pace one or both of the ventricles, switch circuitry 702 may couple first electrode 112, which has penetrated to wall tissue of a ventricle or the intraventricular septum, to signal generation circuitry 706 as a cathode, and one or both of second electrode 114 or electrode 218 to signal generation circuitry 706 as an anode. As another example, in order to pace the RA, switch circuitry 702 may couple second electrode 114, which maintains contact with the RA endocardium, to signal generation circuitry 706 as a cathode, and one or both of first electrode 112 or electrode 218 to signal generation circuitry 706 as an anode.

Each of electrodes 112, 114, 218 may be coupled to switch circuitry 702 via a corresponding feedthrough assembly. In some examples, each feedthrough assembly is substantially straight (e.g., along longitudinal axis 210). In some examples, such as when distal end 204 of housing 202 is removable from housing 202 (e.g., when distal end 204 is a removable header), the feedthrough assemblies are offset to allow for removal of distal end 204. For example, when a header defining distal end 204 is configured to be removably secured to housing 202 (e.g., via a turn-lock mechanism), the feedthrough assemblies are offset from longitudinal axis 210 to allow the header to turn relative to housing 202.

Switch circuitry 702 may also selectively couple sensing circuitry 704 to selected combinations of electrodes 112, 114, and 218, e.g., to selectively sense the electrical activity of either the RA or ventricles of heart 102. Sensing circuitry 704 may include filters, amplifiers, analog-to-digital converters, or other circuitry configured to sense cardiac electrical signals via electrodes 112, 114, and/or 218. For example, switch circuitry 702 may couple each of first electrode 112 and second electrode 114 (in combination with electrode 218) to respective sensing channels provided by sensing circuitry 704 to respectively sense either ventricular or atrial cardiac electrical signals. In some examples, sensing circuitry 704 is configured to detect events, e.g., depolarizations, within the cardiac electrical signals, and provide indications thereof to processing circuitry 710. In this manner, processing circuitry 710 may determine the timing of atrial and ventricular depolarizations, and control the delivery of cardiac pacing, e.g., AV synchronized cardiac pacing, based thereon. Processing circuitry 710 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 710 herein may be embodied as firmware, hardware, software or any combination thereof.

Sensor(s) 708 may include one or more sensing elements that transduce patient physiological activity to an electrical signal to sense values of a respective patient parameter. Sensor(s) 708 may include one or more accelerometers, optical sensors, chemical sensors, temperature sensors, pressure sensors, or any other types of sensors. Sensor(s) 708 may output patient parameter values that may be used as feedback to control sensing and delivery of therapy by device 104.

Telemetry circuitry 712 supports wireless communication between device 104 and an external programmer (not shown in FIG. 7) or another computing device under the control of processing circuitry 710. Processing circuitry 710 of device 104 may receive, as updates to operational parameters from the computing device, and provide collected data, e.g., sensed heart activity or other patient parameters, via telemetry circuitry 712. Telemetry circuitry 712 may accomplish communication by radiofrequency (RF) communication techniques, e.g., via an antenna (not shown).

Power source 716 delivers operating power to various components of device 104. Power source 716 may include a rechargeable or non-rechargeable battery and a power generation circuit to produce the operating power. Recharging may be accomplished through proximal inductive interaction between an external charger and an inductive charging coil within device 104.

While FIGS. 1-7 describe device 104 as configured to be implanted wholly within heart 102, the same structures and components described herein may be used to fix another implantable medical device within tissue of a patient. For example, an implantable medical device may include a fixation device similar to structure and/or function to helix and/or coil defined by first electrode 112 and a fixation mesh (e.g., fixation mesh 222, 226, 404) as described above. In such examples, the fixation mesh may prevent or inhibit unintended rotation of a fixation helix and/or coil extending from a distal end of the fixation device.

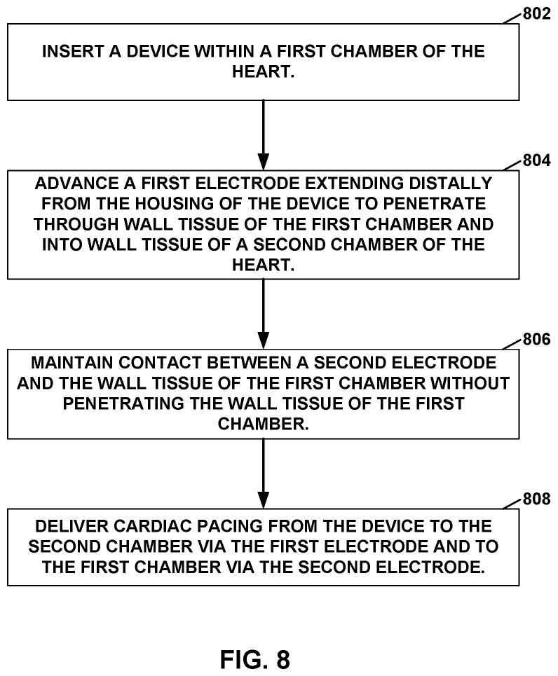

FIG. 8 is a flow diagram illustrating an example process for sensing a cardiac electrical signal and delivering cardiac pacing therapy to a heart of a patient via an device 104 of any of FIGS. 1-7. The technique of FIG. 8 will be described with concurrent reference to device 104 as illustrated in FIGS. 1-7, although a person having ordinary skill in the art will understand that the technique may be performed in reference to an implantable medical lead or other medical device.

A clinician may insert device 104 within a single first chamber of the heart 102 (802). The first chamber of heart 102 may be the right atrium, left atrium, the right ventricle, or the left ventricle. The clinician may insert device 104 into the first chamber via delivery tool connected to device 104 (e.g., connected to delivery tool interface member 208). The clinician may advance first electrode 112 extending distally from housing 202 of device 104 to penetrate through wall tissue of the first chamber and into wall tissue of a second chamber of heart 102 (804). In some examples, advancing first electrode 112 includes positioning a distal end of first electrode 112 (e.g., a first electrically active region 216) within a ventricular myocardium 108 of the patient. The clinician may advance first electrode 112 by rotating device 104 clockwise or counterclockwise within the first chamber, depending on how first electrode 112 is wound.

The clinician may cause device 104 to maintain contact between second electrode 114 and the wall tissue of the first chamber, without penetrating the wall tissue of the first chamber (806). The clinician may advance device 104 into the wall tissue until the wall tissue contacts second electrode 114, protrusion 212 and/or face 205 of housing 202. In some examples, when second electrode 114 is disposed on a protrusion 212 defining a ramp, the ramp extends second electrode 114 distally from face 205 (e.g., from distal end 204) and along longitudinal axis 210 by a fixed distance. Protrusion 212 may cause second electrode 114 to be placed relatively deeper within wall tissue than face 205 without penetrating the wall tissue, thereby allowing the wall tissue to at least partially envelop a distal surface and/or sides of second electrode 114. In some examples, protrusion 212 causes second electrode 114 to maintain contact with the wall tissue of the first chamber (e.g., the atrial endocardium of the patient). Protrusion 212 may place second electrode 114 in consistent contact with the wall tissue of the first chamber, thereby increasing the consistency of the sensing and/or pacing functionalities of second electrode 114.

While device 104 is implanted within the cardiac tissue, device 104 may allow cardiac tissue to grow into a fixation mesh (e.g., fixation mesh 222, 226, 404) disposed on distal end 204 of device 104. The fixation mesh may be disposed over face 205 and/or around an outer perimeter of housing 202. The cardiac tissue may grow into openings 230, into spaces 506 between overlapping filaments 504 of filaments 224 forming the fixation mesh, and/or around individual filaments of the fixation mesh. A therapeutic substance disposed around at least some of filaments 224 may reduce inflammation of the cardiac tissue when fixation mesh is placed in contact with and/or is within the cardiac tissue. The ingrown cardiac tissue may act against filaments 224 of the fixation mesh to inhibit unintended rotation of device 104 within the cardiac tissue. Filaments 224 may be formed from a non-absorbable material and may remain within the cardiac tissue, e.g., to facilitate chronic fixation of device 104 within the cardiac tissue. Filaments 224 may be formed from an absorbable material and may be completely absorbed by the cardiac tissue after a period of time. Once filaments 224 are completely absorbed, the clinician may remove device 104 from the cardiac tissue by rotating device 104 (e.g., via delivery tool interface member 208).

The clinician may deliver cardiac pacing from device 104 to the second chamber via first electrode 112 and to the first chamber via second electrode 114 (808). Device 104 may deliver cardiac pacing to the first chamber and/or the second chamber via first electrode 112, second electrode 114, and/or one or more other electrodes of device 104 (e.g., electrode 218).

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

In addition, it should be noted that system described herein may not be limited to treatment of a human patient. In alternative examples, the system may be implemented in non-human patients, e.g., primates, canines, equines, pigs, and felines. These other animals may undergo clinical or research therapies that may benefit from the subject matter of this disclosure.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

This disclosure describes each of the following examples.

Example 1: a device comprising: an elongated housing extending from a proximal end to a distal end, the elongated housing being configured to be implanted wholly within a chamber of a heart; a first electrode extending distally from the distal end of the elongated housing, the first electrode comprising an elongated body defining a helix; a second electrode disposed on the distal end of the elongated housing, wherein the second electrode is configured to be placed in contact with wall tissue of the chamber without penetrating the wall tissue; and a mesh disposed over at least a portion of a distal portion of the elongated housing, wherein the distal portion of the elongated housing defines the distal end of the housing, wherein the mesh comprises a plurality of filaments, and wherein the mesh is configured to facilitate growth of the wall tissue around one or more filaments of the plurality of filaments.

Example 2: the device of example 1, wherein the mesh is disposed around an outer perimeter of the distal portion of the elongated housing.

Example 3: the device of any of examples 1 and 2, wherein the mesh is disposed over at least a portion of a distal face of the distal end of the elongated housing, wherein the first electrode extends distally from the distal face, and wherein the second electrode is disposed on the distal face.

Example 4: the device of example 3, wherein the plurality of filaments defines two or more openings within the mesh, wherein the first electrode is disposed within a first opening of the two or more openings, and wherein the second electrode is disposed within a second opening of the two or more openings.

Example 5: the device of example 4, further comprising: a ramp extending distally from the distal end of the elongated housing, wherein the second electrode is disposed on the ramp, wherein the ramp is configured to separate the second electrode from the distal end of the elongated housing by a fixed distance, and wherein the ramp is disposed within the second opening.

Example 6: the device of any of examples 1-5, wherein the second electrode is configured to disposed over one or more filaments of the plurality of filaments.

Example 7: the device of any of examples 1-6, wherein two or more filaments of the plurality of filaments intersect at an intersection within the mesh, wherein the intersection defines a gap between the mesh and an outer surface of the elongated housing, and wherein the mesh is configured to allow the growth of the wall tissue into the gap.

Example 8: the device of any of examples 1-7, wherein the mesh comprises an adhesive disposed over one or more filaments of the plurality of filaments, and wherein the mesh is configured to be affixed to the distal portion of the elongated housing via the adhesive.

Example 9: the device of any of examples 1-8, wherein the mesh comprises an attachment member disposed around one or more filaments of the plurality of filaments, and wherein the mesh is configured to be affixed to the distal portion of the elongated housing via the attachment member.

Example 10: the device of any of examples 1-9, wherein the distal portion of the elongated housing comprises a removable header, wherein the removable header defines the distal end of the elongated housing and is configured to be removably secured to a proximal portion of the elongated housing, wherein the proximal portion of the elongated housing comprises an inner surface defining an inner volume of the elongated housing, and wherein at least a portion of the mesh is configured to be disposed within the inner volume when the removable header is secured to the proximal portion of the housing.

Example 11: the device of any of examples 1-10, wherein the distal portion of the elongated housing defines a recess extending around an outer perimeter of the distal portion of the elongated housing, and wherein at least a portion of the mesh is configured to be disposed within the recess.

Example 12: the device of any of examples 1-11, wherein each filament of the plurality of filaments comprises a non-absorbable material.

Example 13: the device of any of examples 1-12, wherein each filament of the plurality of filaments comprises a therapeutic substance disposed over an outer surface of the filament.

Example 14: the device of any of examples 1-13, wherein the second electrode defines a recess, and wherein the device further comprises a therapeutic substance dispensing device disposed within the recess.

Example 15: the device of any of examples 1-14, wherein the chamber of the heart comprises a first chamber of the heart, and wherein the helix is configured to penetrate into wall tissue of a second chamber of the heart that is separated from the first chamber of the heart.

Example 16: the device of example 15, wherein the first chamber comprises an atrium of the heart, and wherein the second chamber comprises a ventricle of the heart.

Example 17: the device of any of examples 1-16, wherein the mesh is configured to inhibit unintended rotation of the elongated body within the wall tissue.

Example 18: the device of any of examples 1-17, wherein the distal end of the elongated housing further comprises one or more features configured to inhibit unintended rotation of the elongated body within the wall tissue.

Example 19: the device of any of examples 1-18, wherein the mesh comprises a woven mesh, a braided mesh, a knitted mesh, or a felt mesh.

Example 20: a fixation device comprising: an elongated body extending distally from a distal end of an implantable medical device, the elongated body comprising: a proximal end located at the distal end of the implantable medical device, and a helix extending distally from the proximal end and defining one or more coils, wherein a distal end of the helix is configured to penetrate into tissue of a patient; and a mesh disposed over at least a portion of a distal portion of the implantable medical device, wherein the distal portion of the implantable medical device defines the distal end of the implantable medical device, wherein the mesh comprises a plurality of filaments, and wherein the mesh is configured to facilitate growth of the tissue around one or more filaments of the plurality of filaments.

Example 21: the fixation device of example 20, wherein the mesh is disposed around an outer perimeter of the distal portion of the implantable medical device.

Example 22: the fixation device of any of examples 20 and 21, wherein the mesh is disposed over at least a portion of a distal face of the distal end of the implantable medical device, wherein the proximal end of the elongated body is disposed on the distal face.

Example 23: the fixation device of example 22, wherein the plurality of filaments defines an opening within the mesh, wherein the elongated body is disposed within the opening.

Example 24: the fixation device of any of examples 20-23, wherein two or more filaments of the plurality of filaments intersect at an intersection within the mesh, wherein the intersection defines a gap between the mesh and an outer surface of the implantable medical device, and wherein the mesh is configured to allow the growth of the tissue into the gap.

Example 25: the fixation device of any of examples 20-24, wherein the mesh comprises an adhesive disposed over one or more filaments of the plurality of filaments, and wherein the mesh is configured to be affixed to the distal portion of the implantable medical device via the adhesive.

Example 26: the fixation device of any of examples 20-25, wherein the mesh comprises an attachment member disposed around one or more filaments of the plurality of filaments, and wherein the mesh is configured to be affixed to the distal portion of the implantable medical device via the attachment member.

Example 27: the fixation device of any of examples 20-26, wherein the distal portion of the implantable medical device comprises a removable member, wherein the removable member defines the distal end of the implantable medical device and is configured to be removably secured to a housing of the implantable medical device, wherein the housing of the implantable medical device comprises an inner surface defining an inner volume, and wherein at least a portion of the mesh is configured to be disposed within the inner volume when the removable member is secured to the housing.

Example 28: the fixation device of any of examples 20-27, wherein the distal portion of the implantable medical device defines a recess extending around an outer perimeter of the distal portion of the implantable medical device, and wherein at least a portion of the mesh is configured to be disposed within the recess.

Example 29: the fixation device of any of examples 20-28, wherein each filament of the plurality of filaments comprises a non-absorbable material.

Example 30: the fixation device of any of examples 20-29, wherein each filament of the plurality of filaments comprises a therapeutic substance disposed over an outer surface of the filament.

Example 31: the fixation device of any of examples 20-30, wherein the mesh is configured to inhibit unintended rotation of the implantable medical device within the tissue.

Example 32: the fixation device of any of examples 20-31, wherein the distal end of the implantable medical device further comprises one or more features configured to inhibit unintended rotation of the implantable medical device with the tissue.

Example 33: the device of any of examples 20-32, wherein the mesh comprises a woven mesh, a braided mesh, a knitted mesh, or a felt mesh.

Example 34: a method comprising: affixing a first electrode to a distal end of an elongated housing of an implantable medical device, wherein the first electrode is configured to extend distally from the distal end of the elongated housing, wherein the first electrode comprises an elongated body defining a helix, and wherein the implantable medical device is configured to be implanted wholly within a chamber of a heart; affixing a second electrode to the distal end of the elongated housing, wherein the second electrode is configured to be placed in contact with wall tissue of the chamber without penetrating the wall tissue; and affixing a mesh over a distal portion of the elongated housing, wherein the distal portion of the elongated housing defines the distal end of the elongated housing, wherein the mesh comprises a plurality of filaments, and wherein the mesh is configured to facilitate growth of the wall tissue around one or more filaments of the plurality of filaments.

Example 35: the method of example 34, wherein affixing the mesh over the distal portion of the elongated housing further comprises: determining a surface area of the distal portion of the elongated housing; shaping a mesh material to a portion of the mesh material of at least the determined surface area, wherein the mesh material comprises interwoven filaments of the plurality of filaments; disposing the portion of the mesh material over the distal portion of the elongated housing to form the mesh; and securing the mesh to an outer surface of the distal portion of the elongated housing.

Example 36: the method of any of examples 34 and 35, wherein the plurality of filaments defines two or more openings within the mesh, and wherein affixing the mesh over the distal portion the elongated housing comprises: placing the first electrode within a first opening of the two or more openings within the mesh; and placing the second electrode within a second opening of the two or more openings within the mesh.

Example 37: the method of example 36, wherein the implantable medical device further comprises a ramp extending distally from the distal end of the elongated housing, wherein the second electrode is disposed on the ramp, wherein the ramp is configured to place the second electrode in contact with the wall tissue and to separate the second electrode from the distal end of the elongated housing by a fixed distance, and wherein placing the second electrode within the second opening comprises placing the ramp within the second opening within the mesh.

Example 38: the method of any of examples 34-37, wherein affixing the second electrode to the distal end of the elongated housing comprises: disposing the mesh over a distal face of the distal end of the elongated housing; placing the second electrode over the mesh, wherein the second electrode is placed over portions of one or more filaments of the plurality of filaments; and connecting the second electrode to computing circuitry disposed within the elongated housing of the implantable medical device.

Example 39: the method of any of examples 34-38, wherein two or more filaments of the plurality of filaments intersect at an intersection within the mesh, wherein the intersection defines a gap between the mesh and an outer surface of the elongated housing, and wherein the mesh is configured to facilitate the growth of the wall tissue into the gap.

Example 40: the method of any of examples 34-39, wherein the mesh comprises an adhesive disposed over one or more filaments of the plurality of filaments, and wherein affixing the mesh over the distal portion of the elongated housing comprises: placing the mesh over the distal portion of the elongated housing; and securing the one or more filaments of the plurality of filaments to an outer surface of the distal portion of the elongated housing via the adhesive.

Example 41: the method of any of examples 34-40, wherein the mesh comprises an attachment member disposed around one or more filaments of the plurality of filaments, and wherein affixing the mesh over the distal portion of the elongated housing comprises: placing the mesh over the distal portion of the elongated housing; aligning the attachment member to a locking feature on an outer surface of the distal portion of the elongated housing; and securing the attachment member to the locking feature.

Example 42: the method of any of examples 34-41, wherein the distal portion of the elongated housing of the implantable medical device comprises a removable header configured to be removably secured to a proximal portion of the elongated housing, wherein the removable header defines the distal end of the elongated housing, and wherein affixing the mesh over the distal portion of the elongated housing comprises: disposing the mesh over the removable header; placing a portion of the mesh extending proximally from the removable header within an inner volume of the proximal portion of the elongated housing; and affixing the removable header to the proximal portion of the elongated housing while the portion of the mesh is disposed within the inner volume of the proximal portion of the elongated housing.

Example 43: the method of any of examples 34-42, wherein the distal portion of the elongated housing defines a recess extending around an outer perimeter of the distal portion of the elongated housing, and wherein affixing the mesh over the distal portion of the elongated housing comprises securing a portion of the mesh within the recess.

Example 44: the method of any of examples 34-43, wherein each filament of the plurality of filaments comprises a non-absorbable material.

Example 45: the method of any of examples 34-44, wherein each filament of the plurality of filaments comprises a therapeutic substance disposed over an outer surface of the filament.

Example 46: the method of any of examples 34-45, wherein the chamber of the heart comprises a first chamber of the heart, and wherein the helix is configured to penetrate into wall tissue of a second chamber of the heart that is separated from the first chamber of the heart.

Example 47: the method of example 46, wherein the first chamber comprises an atrium of the heart, and wherein the second chamber comprises a ventricle of the heart.

Example 48: the method of any of examples 34-47, wherein the mesh is configured to inhibit unintended rotation of the elongated body within the wall tissue.

Example 49: the device of any of examples 34-48, wherein the mesh comprises a woven mesh, a braided mesh, a knitted mesh, or a felt mesh.

Example 50: a device comprising: an elongated housing extending from a proximal end to a distal end, the elongated housing being configured to be implanted wholly within a chamber of a heart; a fixation helix extending distally from the distal end of the elongated housing; an electrode disposed at the distal end of the elongated housing, wherein the second electrode is configured to be placed in contact with wall tissue of the chamber without penetrating the wall tissue; and a mesh disposed over at least a portion of a distal portion of the elongated housing, wherein the distal portion of the elongated housing defines the distal end of the housing, wherein the mesh comprises a plurality of filaments, and wherein the mesh is configured to facilitate growth of the wall tissue around one or more filaments of the plurality of filaments.

Example 51: the device of example 50, wherein the mesh is disposed around an outer perimeter of the distal portion of the elongated housing.

Example 52: the device of any of examples 50 and 51, wherein the mesh is disposed over at least a portion of a distal face of the distal end of the elongated housing, wherein the first electrode extends distally from the distal face, and wherein the second electrode is disposed on the distal face.

Example 53: the device of example 52, wherein the plurality of filaments defines two or more openings within the mesh, wherein the fixation helix is disposed within a first opening of the two or more openings, and wherein the electrode is disposed within a second opening of the two or more openings.

Example 54: the device of any of examples 50-53, wherein two or more filaments of the plurality of filaments intersect at an intersection within the mesh, wherein the intersection defines a gap between the mesh and an outer surface of the elongated housing, and wherein the mesh is configured to allow the growth of the wall tissue into the gap.

Example 55: the device of any of examples 50-54, wherein the mesh comprises an adhesive disposed over one or more filaments of the plurality of filaments, and wherein the mesh is configured to be affixed to the distal portion of the elongated housing via the adhesive.

Example 56: the device of any of examples 50-55, wherein the mesh comprises an attachment member disposed around one or more filaments of the plurality of filaments, and wherein the mesh is configured to be affixed to the distal portion of the elongated housing via the attachment member.

Example 57: the device of any of examples 50-56, wherein the distal portion of the elongated housing comprises a removable header, wherein the removable header defines the distal end of the elongated housing and is configured to be removably secured to a proximal portion of the elongated housing, wherein the proximal portion of the elongated housing comprises an inner surface defining an inner volume of the elongated housing, and wherein at least a portion of the mesh is configured to be disposed within the inner volume when the removable header is secured to the proximal portion of the housing.

Example 58: the device of any of examples 50-57, wherein the distal portion of the elongated housing defines a recess extending around an outer perimeter of the distal portion of the elongated housing, and wherein at least a portion of the mesh is configured to be disposed within the recess.

Example 59: the device of any of examples 50-58, wherein the mesh comprises a woven mesh, a braided mesh, a knitted mesh, or a felt mesh.

Various examples have been described. These and other examples are within the scope of the following claims.

1. A device comprising:
an elongated housing extending from a proximal end to a distal end, the elongated housing being configured to be implanted wholly within a chamber of a heart;
a first electrode extending distally from the distal end of the elongated housing, the first electrode comprising an elongated body defining a helix;
a second electrode disposed on the distal end of the elongated housing, wherein the second electrode is configured to be placed in contact with wall tissue of the chamber without penetrating the wall tissue; and
a mesh disposed over at least a portion of a distal portion of the elongated housing, wherein the distal portion of the elongated housing defines the distal end of the housing, wherein the mesh comprises a plurality of filaments, and wherein the mesh is configured to facilitate growth of the wall tissue around one or more filaments of the plurality of filaments.

2. The device of claim 1, wherein the mesh is disposed around an outer perimeter of the distal portion of the elongated housing.

3. The device of claim 1, wherein the mesh is disposed over at least a portion of a distal face of the distal end of the elongated housing, wherein the first electrode extends distally from the distal face, and wherein the second electrode is disposed on the distal face.

4. The device of claim 3, wherein the plurality of filaments defines two or more openings within the mesh, wherein the first electrode is disposed within a first opening of the two or more openings, and wherein the second electrode is disposed within a second opening of the two or more openings.

5. The device of claim 1, wherein two or more filaments of the plurality of filaments intersect at an intersection within the mesh, wherein the intersection defines a gap between the mesh and an outer surface of the elongated housing, and wherein the mesh is configured to allow the growth of the wall tissue into the gap.

6. The device of claim 1, wherein each filament of the plurality of filaments comprises a therapeutic substance disposed over an outer surface of the filament.

7. The device of claim 1, wherein the chamber of the heart comprises a first chamber of the heart, and wherein the helix is configured to penetrate into wall tissue of a second chamber of the heart that is separated from the first chamber of the heart.

8. The device of claim 1, wherein the mesh is configured to inhibit unintended rotation of the elongated body within the wall tissue.

9. The device of claim 1, wherein the mesh comprises a woven mesh, a braided mesh, a knitted mesh, or a felt mesh.

10. A fixation device comprising:
an elongated body extending distally from a distal end of an implantable medical device, the elongated body comprising:
a proximal end located at the distal end of the implantable medical device, and
a helix extending distally from the proximal end and defining one or more coils, wherein a distal end of the helix is configured to penetrate into tissue of a patient; and
a mesh disposed over at least a portion of a distal portion of the implantable medical device, wherein the distal portion of the implantable medical device defines the distal end of the implantable medical device, wherein the mesh comprises a plurality of filaments, and wherein the mesh is configured to facilitate growth of the tissue around one or more filaments of the plurality of filaments.

11. The fixation device of claim 10, wherein the mesh is disposed around an outer perimeter of the distal portion of the implantable medical device.

12. The fixation device of claim 10, wherein the mesh is disposed over at least a portion of a distal face of the distal end of the implantable medical device, wherein the proximal end of the elongated body is disposed on the distal face.

13. The device of any claim 10, wherein the mesh comprises a woven mesh, a braided mesh, a knitted mesh, or a felt mesh.

14. A device comprising:
an elongated housing extending from a proximal end to a distal end, the elongated housing being configured to be implanted wholly within a chamber of a heart;
a fixation helix extending distally from the distal end of the elongated housing;
an electrode disposed at the distal end of the elongated housing, wherein the second electrode is configured to be placed in contact with wall tissue of the chamber without penetrating the wall tissue; and
a mesh disposed over at least a portion of a distal portion of the elongated housing, wherein the distal portion of the elongated housing defines the distal end of the housing, wherein the mesh comprises a plurality of filaments, and wherein the mesh is configured to facilitate growth of the wall tissue around one or more filaments of the plurality of filaments.

15. The device of claim 14, wherein the mesh comprises a woven mesh, a braided mesh, a knitted mesh, or a felt mesh.

16. The device of claim 14, wherein the mesh is disposed around an outer perimeter of the distal portion of the elongated housing.

17. The device of claim 14, wherein the mesh is disposed over at least a portion of a distal face of the distal end of the elongated housing, wherein the first electrode extends distally from the distal face, and wherein the second electrode is disposed on the distal face.

18. The device of claim 17, wherein the plurality of filaments defines two or more openings within the mesh, wherein the fixation helix is disposed within a first opening of the two or more openings, and wherein the electrode is disposed within a second opening of the two or more openings.

19. The device of claim 14, wherein two or more filaments of the plurality of filaments intersect at an intersection within the mesh, wherein the intersection defines a gap between the mesh and an outer surface of the elongated housing, and wherein the mesh is configured to allow the growth of the wall tissue into the gap.

20. The device of claim 14, wherein the distal portion of the elongated housing defines a recess extending around an outer perimeter of the distal portion of the elongated housing, and wherein at least a portion of the mesh is configured to be disposed within the recess.
